SEQUENCE LISTING (3 sequences)

SEQ ID NO: 1 - protein, 392 aa, Bacillus subtilis
MKKYHISMINIPAYGHVNPTLALVEKLCEKGHRVTYATTEEFAPAVQQAGGEALIYHTSLNIDPKQIREMMEKNDAPLSLLKESLSILPQLEELYKDDQPDLIIYDFVALAGKLFAEKLNVPVIKLCSSYAQNESFQLGNEDMLKKIREAEAEFKAYLEQEKLPAVSFEQLAVPEALNIVFMPKSFQIQHETFDDRFCFVGPSLGERKEKESLLIDKDDRPLMLISLGTAFNAWPEFYKMCIKAFRDSSWQVIMSVGKTIDPESLEDIPANFTIRQSVPQLEVLEKADLFISHGGMNSTMEAMNAGVPLVVIPQMYEQELTANRVDELGLGVYLPKEEVTVSSLQEAVQAVSSDQELLSRVKNMQKDVKEAGGAERAAAEIEAFMKKSAVPQ

SEQ ID NO: 2 - protein, 415 aa, Streptomyces antibioticus
MTTQTTPAHIAMFSIAAHGHVNPSLEVIRELVARGHRVTYAIPPVFADKVAATGARPVLYHSTLPGTDADPEAWGSTLLDNVEPFLNDAIQALPQLADAYADDIPDLVLHDITSYPARVLARRWGVPAVSLFPNLVAWKGYEEEVAEPMWREPRQTERGRAYYARFEAWLKENGITEHPDTFASHPPRSLVLIPKALQPHADRVDEDVYTFVGACQGDRAEEGGWQRPAGAEKVVLVSLGSVFTKQPAFYRECVRAFGNLPGWHLVLQIGRKVTPAELGELPDNVEVHDWVPQLAILRQADLFVTHAGAGGSQEGLATATPMIAVPQAVDQFGNADMLQGLGVARKLATEEATADLLRETALALVDDPEVARRLRRIQAEMAQEGGTRRAADLIEAELPARHERQEPVGDRPNGG

SEQ ID NO: 3 - protein, 396 aa, Bacillus licheniformis
MGHKHIAIFNIPAHGHINPTLALTASLVKRGYRVTYPVTDEFVKAVEETGAEPLNYRSTLNIDPQQIRELMKNKKDMSQAPLMFIKEMEEVLPQLEALYENDKPDLILFDFMAMAGKLLAEKFGIEAVRLCSTYAQNEHFTFRSISEEFKIELTPEQEDALKNSNLPSFNFEDMFEPAKLNIVFMPRAFQPYGETFDERFSFVGPSLAKRKFQEKETPIISDSGRPVMLISLGTAFNAWPEFYHMCIEAFRDTKWQVIMAVGTTIDPESFDDIPENFSIHQRVPQLEILKKAELFITHGGMNSTMEGLNAGVPLVAVPQMPEQEITARRVEELGLGKHLQPEDTTAASLREAVSQTDGDPHVLKRIQDMQKHIKQAGGAEKAADEIEAFLAPAGVK

(19) United States
(12) Patent Application Publication
(10) Pub. No.: US 2023/0193336 A1
(43) Pub. Date: Jun. 22, 2023

(54) ENASIDENIB GLYCOSIDES AND METHODS OF TREATING DISEASES ASSOCIATED WITH ISOCITRATE DEHYDROGENASE (IDH) DYSFUNCTION

(71) Applicant: Double Rainbow Biosciences Inc., Lexington, MA (US)

(72) Inventors: Sheng Ding, Bethany, CT (US); Yasmin-Pei Kamal Chau, Cambridge, MA (US); Jacob Donald Stanley Wirth, Waltham, MA (US); Tian Xu, Guilford, CT (US); Jing-Ke Weng, Belmont, MA (US)

(21) Appl. No.: 17/906,374

(22) PCT Filed: Mar. 15, 2021
(86) PCT No.: PCT/US2021/022410

§ 371 Date: Sep. 15, 2022

Related U.S. Application Data
(60) Provisional application No. 62/990,114, filed on Mar. 16, 2020.

(51) Int. Cl. C12P 19/44; A61K 47/54

(52) U.S. Cl. CPC C12P 19/445; A61K 47/549

(57) ABSTRACT

Enasidenib glycosides and methods of making enasidenib glycosides are disclosed. Glycosyl transferases catalyze addition of one or more monosaccharides to enasidenib to yield enasidenib glycosides. Suitable monosaccharides can be in the L- or D-configuration and typically have 5, 6, or 7 carbons. Suitable monosaccharides include allose, apiose, arabinose, fructose, fucitol, fucose, galactose, galacturonate, glucose, glucuronic acid, mannose, N-acetylglucosamine, rhamnose, or xylose. Uridine diphosphate glycosyl transferases can catalyze formation of either an alpha or beta glycosidic bond.

### RELATED APPLICATION

This application claims the benefit of U.S. Provisional Application No. 62/990,114, filed on Mar. 16, 2020. The entire teachings of the above application are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 57671001001SequenceListing.txt; created Mar. 15, 2021, 11 KB in size.

### BACKGROUND

Acute myeloid leukemia (AML) is a blood cancer characterized by infiltration of the blood, bone marrow, and other tissues with abnormal, proliferative hematopoietic cells (Döhner, Weisdorf, and Bloomfield 2015). It is one of most common types of leukemias in adults, accounting for 32% of all adult leukemia diagnosis (American Cancer Society 2020).

### SUMMARY

Described herein are enasidenib derivatives containing specific monosaccharide(s) or oligosaccharides(s) and methods of making these molecules utilizing enzyme catalysis. Compared to enasidenib, the enasidenib glycosides exhibit increased water solubility, which may contribute to improved pharmacokinetic and/or pharmacodynamic profiles. The compounds may act as prodrugs of enasidenib. The compounds may exhibit improvements in potency towards inhibiting the activity of the mutant isocitrate dehydrogenase (IDH) protein. The compounds may exhibit enhanced therapeutic effects on acute myeloid leukemia (AML) and other diseases associated with IDH dysfunction.

Described herein are compounds represented by the following structural formula:

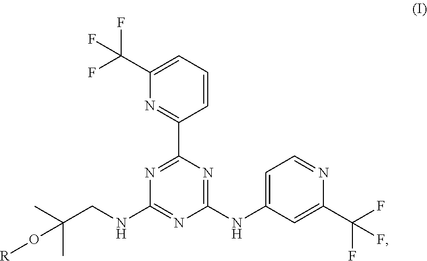

or a pharmaceutically acceptable salt thereof, wherein R is a monosaccharide, a disaccharide, a trisaccharide, or an oligosaccharide having 4 to 10 monosaccharides.

Described herein are pharmaceutical compositions that include an enasidenib glycoside, or a pharmaceutically acceptable salt thereof, and a pharmaceutically acceptable carrier or adjuvant.

Described herein are methods of making an enasidenib glycoside. The methods include: a) providing a reaction mixture; and b) allowing the reaction mixture to convert enasidenib to a monosaccharide, disaccharide, or oligosaccharide of enasidenib. The reaction mixture can include a compound having the following structural formula:

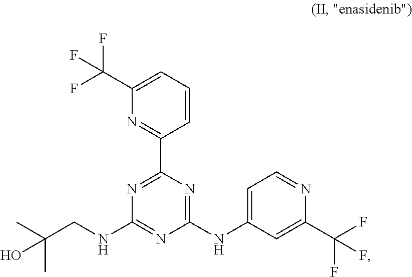 [SMILES: CC(C)(O)CNc1nc(Nc2ccnc(C(F)(F)F)c2)nc(-c2cccc(C(F)(F)F)n2)n1]

a uridine diphosphate glycosyltransferase (UGT); and uridine diphosphate-monosaccharide.

In some embodiments, R is a monosaccharide. In some embodiments, the monosaccharide is a pentose monosaccharide, hexose monosaccharide, or heptose monosaccharide.

In some embodiments, R is allose, apiose, arabinose, fructose, fucitol, fucose, galactose, glucose, glucuronic acid, mannose, N-acetylglucosamine, N-acetylgalactosamine, rhamnose, or xylose. In some embodiments, R is glucosamine, galactosamine, mannosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, or D-glycero-D-altro-heptitol.

In some embodiments, R is a disaccharide. In some embodiments, R is a disaccharide of two glucose molecules. In some embodiments, R is a disaccharide of two galactose molecules. In some embodiments, R is a disaccharide of two xylose molecules. For any of the foregoing disaccharides, the disaccharide molecules can be bonded by a 1→3 glycosidic bond.

In some embodiments, R is a trisaccharide. In some embodiments, R is a trisaccharide of three glucose molecules. In some embodiments, R is a trisaccharide of three galactose molecules. In some embodiments, R is a trisaccharide of three xylose molecules. For any of the foregoing trisaccharides, the trisaccharide molecules can be bonded by a 1→3 glycosidic bond and by a 1→2 glycosidic bond. For any of the foregoing trisaccharides, the trisaccharide molecules can be bonded by a 1→3 glycosidic bond and by a 1→4 glycosidic bond.

In some embodiments, the UGT includes an amino acid sequence that is at least 95% similar to SEQ ID NO: 1. In some embodiments, the UGT includes an amino acid sequence that is at least 80% similar to a region from V278 to Q318 of SEQ ID NO: 1. In some embodiments, the UGT includes an amino acid sequence that is: at least 90% similar to a region from 167 to D75 of SEQ ID NO: 1; at least 90% similar to a region from D106 to L114 of SEQ ID NO: 1; at least 90% similar to a region from C127 to S129 of SEQ ID NO: 1; and at least 80% similar to a region from V278 to Q318 of SEQ ID NO: 1.

In some embodiments, the UGT includes an amino acid sequence that is at least 95% similar to SEQ ID NO: 2. In some embodiments, the UGT includes an amino acid sequence that is at least 80% similar to a region from V291 to Q331 of SEQ ID NO: 2. In some embodiments, the UGT includes an amino acid sequence that is: at least 90% similar to a region from W74 to V82 of SEQ ID NO: 2; at least 90% similar to a region from D111 to V119 of SEQ ID NO: 2; at least 90% similar to a region from F132 to N134 of SEQ ID NO: 2; and at least 80% similar to a region from V291 to Q331 of SEQ ID NO: 2.

In some embodiments, the UGT includes an amino acid sequence that is at least 95% similar to SEQ ID NO: 3. In some embodiments, the UGT includes an amino acid sequence that is at least 80% similar to a region from V283 to Q323 of SEQ ID NO: 3. In some embodiments, the UGT includes an amino acid sequence that is: at least 90% similar to a region from I67 to Q79 of SEQ ID NO: 3; at least 90% similar to a region from D110 to L118 of SEQ ID NO: 3; at least 90% similar to a region from C131 to T133 of SEQ ID NO: 3; and at least 80% similar to a region from V283 to Q323 of SEQ ID NO: 3.

In some embodiments, the uridine diphosphate-monosaccharide is uridine diphosphate-glucose (“UDP-glucose”), uridine diphosphate-galactose (“UDP-galactose”), uridine diphosphate-xylose (“UDP-xylose”), or uridine diphosphate-N-acetylglucosamine (“UDP-N-acetylglucosamine”).

Described herein are methods of treating acute myeloid leukemia or an isocitrate dehydrogenase related disease. The method can include administering to a patient in need thereof a therapeutically effective amount of a compound having the following structural formula:

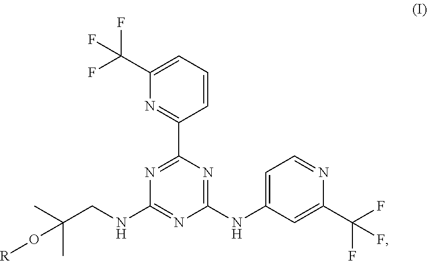

or a pharmaceutically acceptable salt thereof, wherein R is a monosaccharide, a disaccharide, a trisaccharide, or an oligosaccharide comprising 4 to 10 monosaccharides.

In some embodiments, the method further includes administering one or more of azacitidine and decitabine to the patient.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows HPLC chromatograms of the UGT screen results using cell lysates from SEQ ID NO: 2 (2: top chromatogram) and empty vector only control (1: bottom chromatogram) when enasidenib was used as substrate and UDP-glucose was used as the sugar donor. The two extra peaks present in the chromatogram of SEQ ID NO: 2 were glycosylated products enasidenib-O-D-glucoside (chromatogram peak a) and enasidenib-di-O-D-glucoside (chromatogram peak b).

FIG. 2 shows HPLC chromatograms of the purified recombinant glycosyltransferase assay results from SEQ ID NO: 2 obtained after 2 days of reaction incubation (3: top chromatogram), 2 hours of reaction incubation (2: middle chromatogram), and a control reaction containing no glycosyltransferase (1: bottom chromatogram) when enasidenib was used as substrate and UDP-glucose was used as the sugar donor. Labeled peaks show enasidenib-O-D-glucoside (chromatogram peak a), enasidenib-di-O-D-glucoside (chromatogram peak b), and enasidenib-tri-O-D-glucoside (chromatogram peak c).

FIG. 3 is a chart showing water solubility of enasidenib and enasidenib-O-D-glucoside.

FIG. 4 is a multiple sequence alignment of three UGTs (SEQ ID NOs: 1-3) highlighting similar sequence regions important for catalytic function. The PSPG box is underlined. The acceptor binding residues are bolded. Sequence Similarity is defined by positive BLAST similarity using the BLOSUM62 scoring matrix and existent: 11, extension: 1 gap penalties.

FIG. 5 shows 3D structures of UGTs indicating the sequence regions that are important for substrate and/or donor binding. All substrates are colored with black carbon sticks (oxygen=red, nitrogen=blue, phosphorous=orange). Cartoon proteins are rainbow from N- to C-terminus. Center: A global structural superposition comprised of multiple UGT crystal structures and homology models. As labeled, zoomed-in regions are clockwise from top-right: W74-V82, F132-N134, V291-Q331, D111-I119. All numbering follows the sequence of SEQ ID NO: 2 with relevant amino acids shown as sticks.

### DETAILED DESCRIPTION

A description of example embodiments follows.

Acute Myeloid Leukemia (AML) and Other Diseases Associated with Isocitrate Dehydrogenase (IDH) Dysfunction

Mortality rates for AML patients have not changed since 2008. The 5-year survival rate for pediatric patients and adults less than 60 years old is approximately 50%. Due to a variety of factors including increased comorbidities and more complicated and adverse genetic lesions, survival for older AML patients greater than 60 years old decreases substantially to less than 10% (Alibhai et al. 2009; Oran and Weisdorf 2012; Walter and Estey 2015). Survival rates for patients with relapsed or refractory AML is similarly poor with a 5-year survival rate of less than 10% (Stein et al. 2017). As a result, there is a need for new AML therapeutics.

AML occurs as a result of multiple genetic and/or epigenetic lesions that affect cellular proliferation, differentiation, and apoptosis. Because of the heterogeneity of the underlying molecular pathways that are misregulated in AML, it is a difficult cancer to treat successfully. The most common treatment for AML is cytotoxic chemotherapy with cytarabine and an anthracycline, although other chemotherapeutics such as etoposide may also be used (Döhner et al. 2010). Additional therapeutics including the hypomethylating agents azacitidine and decitabine may be used in combination or individually (Thol et al. 2015). Post-remission treatment includes prolonged maintenance therapies utilizing cytotoxic chemotherapeutics or allogeneic hematopoietic stem cell transplantation.

Mutations in the isocitrate dehydrogenase (IDH) gene are found in approximately 20% of AML patients, and occur more frequently as patient age increases (Medeiros et al. 2017; Döhner et al. 2018). IDH is a homodimeric enzyme that catalyzes the NADP-dependent oxidative decarboxylation of isocitrate to alpha-ketoglutarate, and plays a key role in the citric acid or Kreb's cycle as well as other pathways that utilize alpha-ketoglutarate. Two isoforms of IDH are known to acquire oncogenic mutations: IDH1 is localized to the cytoplasm and is associated more frequently with solid tumors, and IDH2 is localized to the mitochondria and is associated more frequently with hematological cancers such as AML (Stein 2016). IDH2 regulates the cellular response to hypoxia as well as the activity of histone demethylases and methylcytosine dioxygenases, both of which are important regulators of the DNA epigenetic landscape. Alpha-ketoglutarate is utilized as a substrate or cofactor by various enzymes such as dioxygenases and demethylases to control many cellular processes including DNA methylation.

The most common AML-associated IDH2 mutations are heterozygous single point mutations of the arginine residue at position 140 or 172 (R140 and R172, respectively). R140 and R172 IDH2 mutants gain a new function: they catalyze the NADPH-dependent reduction of alpha-ketoglutarate to the oncometabolite R-2-hydroxyglutarate (2-HG; sometimes called 2-oxoglutarate).

2-HG is structurally similar to alpha-ketoglutarate, and inhibits many alpha-ketoglutarate-dependent enzymes such as the ten-eleven-translocation (TET) family of 5-methylcytosine dioxygenases and the jumonji-domain-containing family of histone lysine demethylases. As a result, global hypermethylation is a key characteristic found in AML patients with an IDH2 mutation, and contributes to the cancer phenotype by altering the normal regulation of transcription. In vivo and in vitro experiments have shown that increased concentrations of 2-HG lead to increased proliferation and reduced differentiation in a variety of cell types including immature hematopoietic cells. Furthermore, increased concentrations of 2-HG are detected in both cell lines and in human AML patients with mutated IDH2 (Yen et al. 2017).

Other diseases may also be driven by abnormal IDH activity. Targeting IDH1 or IDH2 is a potential therapeutic option for other cancers including glioma, chondrosarcoma, angioimmunoblastic T-cell lymphoma cancers, intrahepatic cholangiocarcinoma, and precancerous diseases such as myelodysplastic syndrome. Other types of diseases including D-2-hydroxyglutaric aciduria and Ollier and Maffucci syndromes are also driven by metabolic imbalances due to abnormal IDH activity (Stein 2016; Huang and Cheng 2017; Waitkus, Diplas, and Yan 2018). Developing inhibitors that can correct these metabolic imbalances is a promising therapeutic option for treating AML and other diseases associated with IDH dysfunction.

### Enasidenib

Enasidenib is a compound represented by the following structural formula:

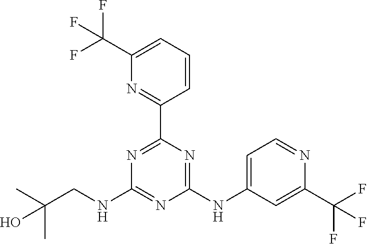 [SMILES: CC(C)(O)CNc1nc(Nc2ccnc(C(F)(F)F)c2)nc(-c2cccc(C(F)(F)F)n2)n1]

Enasidenib (IDHIFA™, AG-221) is an IDH2 small molecule triazine allosteric inhibitor. Enasidenib is not a cytotoxic agent. Instead, its anticancer activity occurs through promotion of differentiation. Enasidenib is approved to treat adult patients with relapsed or refractory acute myeloid leukemia (AML) with a R140 or R172 IDH2 mutation (Celgene Corp 2017). Clinical trials show that treatment with enasidenib as a single therapeutic resulted in an overall response rate of 41% for both AML patients and for relapsed/refractory-AML patients with an IDH2 mutation (Stein et al. 2015, 2017). 19.3% of AML patients with an IDH2 mutation experienced complete remission.

A phase III clinical trial (IDHENTIFY) with enasidenib is currently ongoing to evaluate enasidenib efficacy compared to conventional therapeutics in older patients (NIH US National Library of Medicine n.d.; Galkin and Jonas 2019). Other clinical trials are testing the effects of combining enasidenib treatment with cytotoxic chemotherapeutics or with hypomethylating agents, and of using enasidenib as a maintenance therapeutic after allogeneic hematopoietic stem cell transplantation (Galkin and Jonas 2019). Additional phase I and II clinical trials are ongoing to test enasidenib as a therapeutic for glioma, angioimmunoblastic T-cell lymphoma, intrahepatic cholangiocarcinoma, chondrosarcoma, and myelodysplastic syndrome (NIH US National Library of Medicine n.d.; Galkin and Jonas 2019).

The mechanism of action of IDH2 inhibition by enasidenib has been explored in great depth (Yen et al. 2017). Enasidenib inhibits IDH2 R140 and R172 mutant homodimers or heterodimers at approximately 40-fold lower concentrations compared to the wild type IDH2. Kinetics assays show that enasidenib exhibits slow on/slow off tight binding inhibition kinetics. An X-ray crystal structure of IDH2^{R140Q} bound to enasidenib, NADPH, and calcium show that enasidenib binds at an allosteric site on the IDH2 dimer interface. Enasidenib binding stabilizes the IDH2 open conformation, preventing the normal conformational changes required for catalysis. Treatment with enasidenib reduced the 2-HG concentration in cancer cell lines containing endogenous or exogenously expressed mutant IDH2, mouse xenograft models of AML, and in human AML patients (Yen et al. 2017; Fan et al. 2014; Stein et al. 2017). Treatment with enasidenib also induced differentiation in cell culture assays (Yen et al. 2017).

The biophysical characteristics and pharmacokinetics/pharmacodynamics (PK/PD) of enasidenib has also been well explored (Yen et al. 2017; Celgene Corp 2017). Due to the low aqueous solubility of enasidenib (less than 74 μg/mL; 23 μM at pH 7.4 (Yen et al. 2017)), its oral bioavailability is only 57% with a 100 mg dose.

Despite the success seen in clinical trials with enasidenib, enasidenib faces several limitations. Serious adverse events were reported in approximately 77% of patients treated with enasidenib in clinical trials, and 43% of patients in clinical trials had to stop taking the drug as a result of adverse events. 17% of patients completely discontinued enasidenib. The most common side effects reported in clinical trials are differentiation syndrome and nausea (Celgene Corp 2017; Galkin and Jonas 2019). Differentiation syndrome is life threatening and results from rapid proliferation and differentiation of myeloid cells (Fathi et al. 2017). Symptoms include respiratory distress, pulmonary infiltrates, pleural effusion, renal impairment, multi-organ failure. Other side effects include abnormal bilirubin metabolism via inhibition of UGT1A1 by enasidenib, tumor lysis syndrome, noninfectious leukocytosis, and embryo-fetal toxicity.

Enasidenib faces competition from other IDH2 inhibitors and therapeutics targeting the metabolic imbalances characteristic of AML and other cancers. Other IDH2 small molecule inhibitors are in development such as AG-881, which can penetrate the blood-brain barrier and is in clinical trials for the treatment of solid tumors (Mellinghoff et al. 2018). Therapies targeting other aspects of the metabolic imbalance in AML are also in development such as Venetoclax, a BCL-2 inhibitor, which is part of a signaling pathway that is sensitized in AML patients with an IDH2 mutation; Telaglenastat (CB-839), which is a glutaminase inhibitor that blocks the production of glutamine, the primary metabolic source for the production of alpha-ketoglutarate; and all trans-retinoic acid, which promotes cellular differentiation.

Furthermore, acquired resistance to IDH2 inhibition by enasidenib has been described (Intlekofer et al. 2018). De novo single point mutations of IDH2 residues that interact with enasidenib have been identified in patients who experienced a relapse of AML disease after treatment with enasidenib. These mutations lead to a reduction in enasidenib binding affinity, and a subsequent increase in 2-HG concentration.

Given these limitations, there is a need to address the low aqueous solubility and PK/PD characteristics of enasidenib to improve its efficacy in treating AML or other diseases that can benefit from IDH2 inhibition. For example, deuterated derivatives of enasidenib have been described (Huang and Cheng 2017). Pharmacokinetics assays and in vitro metabolic stability assays show that these derivatives exhibit increased accumulation in blood and decreased metabolic degradation into oxidized and dealkylated metabolites compared to enasidenib.

### Glycosylation

A potential strategy for improving or modulating the efficacy, safety, and/or PK/PD profile of a small molecule-based therapeutic such as enasidenib is modification by glycosylation. The small molecule, or aglycone, is modified by the addition of one or more sugar groups or chains of two or more sugar groups (called oligosaccharides) to nucleophilic centers of the aglycone. These sugar groups can be naturally occurring sugars such as glucose, fructose, rhamnose, mannose, galactose, fucose, xylose, arabinose, glucuronic acid, or N-acetylglucosamine, or they can be synthetically synthesized sugars (e.g., 6-Br-D-glucose, 2-deoxy-D-glucose, 5-thio-D-glucose). These sugars can be attached to the small molecule or to other sugar groups by either an alpha or beta glycosidic bond.

In general, glycosylation of a small molecule can lead to increased aqueous solubility, altered interactions with proteins and membranes, altered absorption and excretion, changes in metabolic stability, and other changes in PK/PD characteristics (Gantt, Peltier-Pain, and Thorson 2011; Kr̆en 2008; De Bruyn et al. 2015).

Glycosylation can enhance or block the transport of a glycoside into specific tissues or organs. Glycosylation can enhance uptake through interaction between the glycoside moiety and lectins or glucose transporters on the cell surface.

In some cases, glycosylation alters the pharmacological activity of the drug, either by enhancing or decreasing potency or even by changing the mechanism of action (Kr̆en 2008; Gantt, Peltier-Pain, and Thorson 2011; De Bruyn et al. 2015).

The identity of the sugar and the stereochemistry of the glycosidic bond can also affect the pharmacological activity or PK/PD profile of a glycoside.

Glycosylation is also a potential strategy for developing prodrugs and compounds for targeted drug delivery to specific tissues. Glycosidases are enzymes that catalyze the hydrolysis of glycosidic bonds and that are specifically expressed in different tissues and organs including blood plasma, the colon, the intestines, and the gut microflora. Glycosidases exhibit substrate specificity towards different glycosidic bond stereochemistry or towards different monosaccharides. A glycosylated drug could function as a prodrug or as a targeted drug if it is preferentially cleaved by a tissue-specific glycosidase. This has been demonstrated by Zipp et al: the alpha-glycosidic bonds in cannabinoid glycosides have been shown to be preferentially cleaved by glycosidases present in the large intestine of mice and not by other chemical or enzymatic processes that may be present in the small intestine, stomach, blood plasma, or brain (Zipp, Hardman, and Brooke 2018; Hardman, Brooke, and Zipp 2017).

In summary, glycosylation of a small molecule may improve aqueous solubility, but may also alter interactions with proteins and membranes, pharmacological activity, and/or PK/PD characteristics in ways that are unexpected.

### Glycosyltransferases

Traditional methods for glycosylating small molecules are non-selective, and it is particularly difficult to control the stereo- and regiospecificity of glycosylation (Zhu and Schmidt 2009; Gu et al. 2014). There is often more than one position on the aglycone that will react with the reagent used to make the desired modification. This makes it necessary to chemically ‘block’ or render temporarily unreactive, the other positions on the molecule in order to selectively modify the desired position. A typical modification will require multiple protection and de-protection steps using the standard methods of synthetic organic chemistry.

Glycosyltransferases (GTs) are a class of enzyme with the potential to act as the catalyst for the generation of novel glycosylated therapeutic small molecules. GTs catalyze the transfer of a sugar from an activated sugar donor molecule to an acceptor molecule (Lairson et al. 2008). They are a large and well-characterized family found in viruses, archaea, bacteria, and eukaryotes. Greater than 600,000 GTs categorized into approximately 110 families are described in the Carbohydrate-active Enzymes Directory (www.cazy.org), and greater than 150 GT structures are reported (www.rcsb.org) (Lombard et al. 2014; Berman 2000). The majority of GTs utilize nucleotide-activated sugar donors and are referred to as Leloir GTs, although lipid phosphate and phosphate-activated sugar donors are also used (Breton, Fournel-Gigleux, and Palcic 2012; Lairson et al. 2008). GT acceptors include proteins, lipids, oligosaccharides, and small molecules.

GTs offer several advantages as a potential tool in a general small molecule glycosylation platform (De Bruyn et al. 2015; Gantt, Peltier-Pain, and Thorson 2011; Yonekura-Sakakibara and Hanada 2011; Schmid et al. 2016). GTs are often characterized by very high conversion efficiencies (up to 100%). As a result, lower concentrations of potentially expensive or difficult to synthesize substrates are required for GT-catalyzed reactions. GTs are able to glycosylate a wide variety of acceptor structures, with many GTs exhibiting promiscuity towards the sugar donor and acceptor. Furthermore, GTs can catalyze the formation of O-, N-, S-, and even C-glycosides. As a result of these characteristics, GTs are generally amenable to both in vitro and in vivo bioengineering efforts.

### Uridine Diphosphate GTs (UGTs)

Uridine diphosphate GTs (UGTs) utilize uridine diphosphate (UDP) sugar donors, and form the largest group of Leloir GTs in plants (Yonekura-Sakakibara and Hanada 2011). Recently, the identification and characterization of new UGTs, especially in plants and bacteria, has exploded as part of an increased interest in characterizing natural product biosynthetic pathways. This method is described by Torens-Spence et al. (Torrens-Spence et al. 2018). In this paper, 33 UGT enzyme-encoding genes were cloned from a Golden root plant, expressed in yeast, and screened for regiospecific activity in modifying tyrosol to produce salidroside or icariside D2, which are tyrosol metabolites in the plant's native salidroside biosynthetic pathway. Another group identified naturally occurring enzymes having promiscuous N- and O-glycosyltransferase activity by mining the expressed genes of Carthamus tinctorius. K. Xie et al. (Xie et al. 2017) describes the identification of a promiscuous glycosyltransferase (UGT71E5) from C. tinctorius which contains N-glycosylase activity towards multiple diverse nitrogen-heterocyclic aromatic compounds. Zhang et al. (Zhang et al. 2019) describes the identification of three new UGTs (UGT 84A33, UGT 71AE1 and UGT 90A14) from C. tinctorius having promiscuous O-glycosyltransferase activity against benzylisoquinoline alkaloids and their use in making glycosylated derivatives. With the continuing technological improvements and decreasing costs of genome and transcriptome sequencing and analysis, it is becoming easier to identify and characterize naturally occurring GTs for the development of novel small molecule diversity generating platforms.

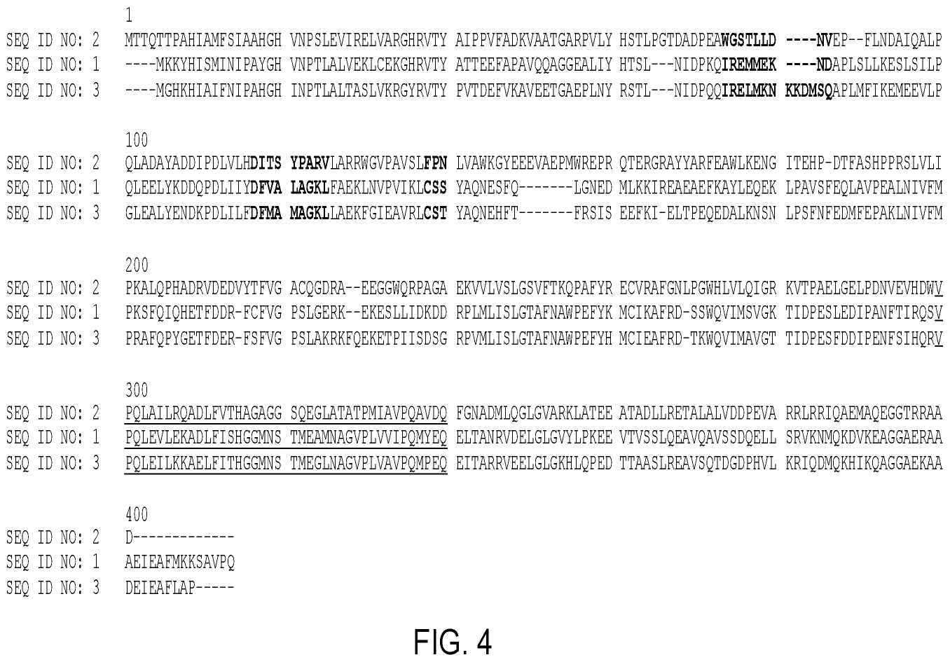

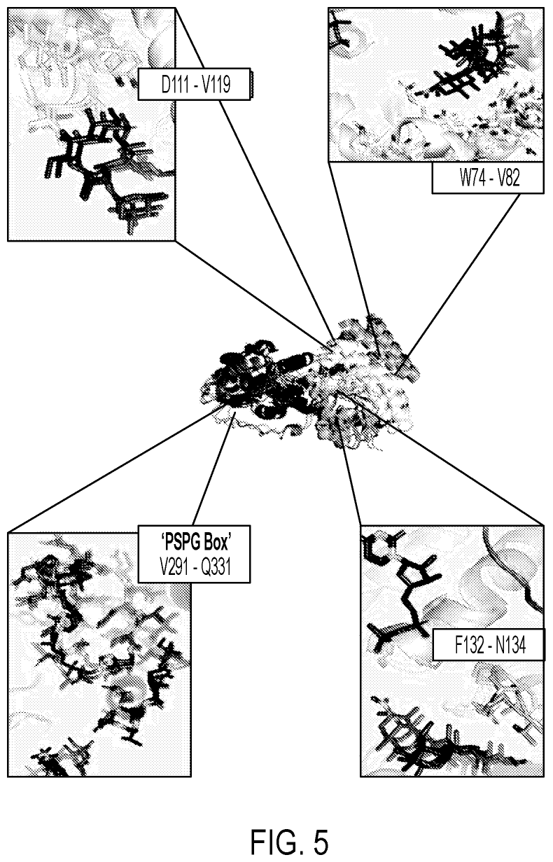

As described herein, four regions within UGT sequences are identified as important for activity. The sequences of all four regions in SEQ ID NO: 1-3 are unique in comparison to other UGTs but highly similar among themselves (FIG. 4). This indicates a strong correlation between the sequences within the four regions and those enzymes' unique activity toward enasidenib. Three acceptor binding sites are shown in crystal structures (or homology models) as poised to interact with sugar acceptor molecules. The “PSPG Box” region is involved in both UGT donor and acceptor substrate affinity and is likely a major part of specific activity (FIG. 5) (Bairoch 1991; Hughes and Hughes 1994; Yamazaki, Gong et al. 1999; Hans, Brandt et al. 2004; Shao, He et al. 2005; He, Wang et al. 2006; Offen, Martinez-Fleites et al. 2006).

TABLE 1
UGT Enzyme Regions Important for Activity

| | | | Sequence |
|---|---|---|---|
| Enzyme | Region | Function | Similarity* |
| SEQ ID NO: 1 | I67 - D75 | Acceptor Substrate Binding | 90% |
| (uridine diphosphate | D106 - L114 | Acceptor Substrate Binding | 90% |
| glycosyltransferase | C127 - S129 | Acceptor Substrate Binding | 90% |
| (UGT) from | V278 - Q318 | “PSPG Box” - Donor/Acceptor Binding | 80% |
| Bacillus subtilis) | | | |
| SEQ ID NO: 2 | W74 - V82 | Acceptor Substrate Binding | 90% |
| (uridine diphosphate | D111 - V119 | Acceptor Substrate Binding | 90% |
| glycosyltransferase | F132 - N134 | Acceptor Substrate Binding | 90% |
| (UGT) from | V291 - Q331 | “PSPG Box” - Donor/Acceptor Binding | 80% |
| Streptomyces antibioticus) | | | |
| SEQ ID NO: 3 | I67 - Q79 | Acceptor Substrate Binding | 90% |
| (uridine diphosphate | D110 - L118 | Acceptor Substrate Binding | 90% |
| glycosyltransferase | C131 - T133 | Acceptor Substrate Binding | 90% |
| (UGT) from | V283 - Q323 | “PSPG Box” - Donor/Acceptor Binding | 80% |
| Bacillus licheniformis) | | | |

*Sequence Similarity is defined by positive BLAST similarity using the BLOSUM62 scoring matrix and existent: 11, extension: 1 gap penalties (Altschul et al. 1990; Henikoff et al. 1992). A commonly used tool for determining percent sequence identity is Protein Basic Local Alignment Search Tool (BLASTp) available through National Center for Biotechnology Information, National Library of Medicine, of the United States National Institutes of Health.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 1. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 1.

In some embodiments, the UGT includes an amino acid sequence that is at least 80% (85%, 90%, 91%, 92%, 93%, 94%, 95%, 96%, 97%, 98%, 99%, or 100%) similar to a region from V278 to Q318 of SEQ ID NO: 1. In some embodiments, the UGT includes an amino acid sequence that is at least 80% (85%, 90%, 91%, 92%, 93%, 94%, 95%, 96%, 97%, 98%, 99%, or 100%) identical to a region from V278 to Q318 of SEQ ID NO: 1

In some embodiments, the UGT includes an amino acid sequence that is: at least 90% (91%, 92%, 93%, 94%, 95%, 96%, 97%, 98%, 99%, or 100%) similar to a region from I67 to D75 of SEQ ID NO: 1; at least 90% (91%, 92%, 93%, 94%, 95%, 96%, 97%, 98%, 99%, or 100%) similar to a region from D106 to L114 of SEQ ID NO: 1; at least 90% (91%, 92%, 93%, 94%, 95%, 96%, 97%, 98%, 99%, or 100%) similar to a region from C127 to S129 of SEQ ID NO: 1; and at least 80% (85%, 90%, 91%, 92%, 93%, 94%, 95%, 96%, 97%, 98%, 99%, or 100%) similar to a region from V278 to Q318 of SEQ ID NO: 1.

In some embodiments, the UGT includes an amino acid sequence that is: at least 90% (91%, 92%, 93%, 94%, 95%, 96%, 97%, 98%, 99%, or 100%) identical to a region from I67 to D75 of SEQ ID NO: 1; at least 90% (91%, 92%, 93%, 94%, 95%, 96%, 97%, 98%, 99%, or 100%) identical to a region from D106 to L114 of SEQ ID NO: 1; at least 90% (91%, 92%, 93%, 94%, 95%, 96%, 97%, 98%, 99%, or 100%) identical to a region from C127 to S129 of SEQ ID NO: 1; and at least 80% (85%, 90%, 91%, 92%, 93%, 94%, 95%, 96%, 97%, 98%, 99%, or 100%) identical to a region from V278 to Q318 of SEQ ID NO: 1.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 2. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 2.

In some embodiments, the UGT includes an amino acid sequence that is at least 80% (85%, 90%, 91%, 92%, 93%, 94%, 95%, 96%, 97%, 98%, 99%, or 100%) similar to a region from V291 to Q331 of SEQ ID NO: 2. In some embodiments, the UGT includes an amino acid sequence that is at least 80% (85%, 90%, 91%, 92%, 93%, 94%, 95%, 96%, 97%, 98%, 99%, or 100%) identical to a region from V291 to Q331 of SEQ ID NO: 2.

In some embodiments, the UGT includes an amino acid sequence that is: at least 90% (91%, 92%, 93%, 94%, 95%, 96%, 97%, 98%, 99%, or 100%) similar to a region from W74 to V82 of SEQ ID NO: 2; at least 90% (91%, 92%, 93%, 94%, 95%, 96%, 97%, 98%, 99%, or 100%) similar to a region from D111 to V119 of SEQ ID NO: 2; at least 90% (91%, 92%, 93%, 94%, 95%, 96%, 97%, 98%, 99%, or 100%) similar to a region from F132 to N134 of SEQ ID NO: 2; and at least 80% (85%, 90%, 91%, 92%, 93%, 94%, 95%, 96%, 97%, 98%, 99%, or 100%) similar to a region from V291 to Q331 of SEQ ID NO: 2.

In some embodiments, the UGT includes an amino acid sequence that is: at least 90% (91%, 92%, 93%, 94%, 95%, 96%, 97%, 98%, 99%, or 100%) identical to a region from W74 to V82 of SEQ ID NO: 2; at least 90% (91%, 92%, 93%, 94%, 95%, 96%, 97%, 98%, 99%, or 100%) identical to a region from D111 to V119 of SEQ ID NO: 2; at least 90% (91%, 92%, 93%, 94%, 95%, 96%, 97%, 98%, 99%, or 100%) identical to a region from F132 to N134 of SEQ ID NO: 2; and at least 80% (85%, 90%, 91%, 92%, 93%, 94%, 95%, 96%, 97%, 98%, 99%, or 100%) identical to a region from V291 to Q331 of SEQ ID NO: 2.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 3. In some embodiments, the UGT includes s an amino acid sequence that is at least 80% (85%, 90%, 91%, 92%, 93%, 94%, 95% 96%, 97%, 98%, 99%, or 100%) identical to SEQ ID NO: 3.

In some embodiments, the UGT includes an amino acid sequence that is at least 80% (85%, 90%, 91%, 92%, 93%, 94%, 95%, 96%, 97%, 98%, 99%, or 100%) similar to a region from V283 to Q323 of SEQ ID NO: 3. In some embodiments, the UGT includes an amino acid sequence that is at least 80% (85%, 90%, 91%, 92%, 93%, 94%, 95%, 96%, 97%, 98%, 99%, or 100%) identical to a region from V283 to Q323 of SEQ ID NO: 3.

In some embodiments, the UGT includes an amino acid sequence that is: at least 90% (91%, 92%, 93%, 94%, 95%, 96%, 97%, 98%, 99%, or 100%) similar to a region from I67 to Q79 of SEQ ID NO: 3; at least 90% (91%, 92%, 93%, 94%, 95%, 96%, 97%, 98%, 99%, or 100%) similar to a region from D110 to L118 of SEQ ID NO: 3; at least 90% (91%, 92%, 93%, 94%, 95%, 96%, 97%, 98%, 99%, or 100%) similar to a region from C131 to T133 of SEQ ID NO: 3; and at least 80% (85%, 90%, 91%, 92%, 93%, 94%, 95%, 96%, 97%, 98%, 99%, or 100%) similar to a region from V283 to Q323 of SEQ ID NO: 3.

In some embodiments, the UGT includes an amino acid sequence that is: at least 90% (91%, 92%, 93%, 94%, 95%, 96%, 97%, 98%, 99%, or 100%) identical to a region from I67 to Q79 of SEQ ID NO: 3; at least 90% (91%, 92%, 93%, 94%, 95%, 96%, 97%, 98%, 99%, or 100%) identical to a region from D110 to L118 of SEQ ID NO: 3; at least 90% (91%, 92%, 93%, 94%, 95%, 96%, 97%, 98%, 99%, or 100%) identical to a region from C131 to T133 of SEQ ID NO: 3; and at least 80% (85%, 90%, 91%, 92%, 93%, 94%, 95%, 96%, 97%, 98%, 99%, or 100%) identical to a region from V283 to Q323 of SEQ ID NO: 3.

### Monosaccharides, Disaccharides, Trisaccharides, and Oligosaccharides

Glycosyltransferases can catalyze the addition of many different monosaccharides to enasidenib. In general, suitable monosaccharides include, but are not limited to, open and closed chain monosaccharides. The monosaccharides can be in the L- or D-configuration. Typically, the monosaccharides have 5, 6, or 7 carbons (a pentose monosaccharide, hexose monosaccharide, or heptose monosaccharide, respectively).

Suitable monosaccharides include allose, apiose, arabinose, fructose, fucitol, fucose, galactose, glucose, glucuronic acid, mannose, N-acetylglucosamine, N-acetylgalactosamine, rhamnose, and xylose. Other suitable monosaccharides include glucosamine, galactosamine, mannosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, and D-glycero-D-altro-heptitol.

Suitable oligosaccharides include, but are not limited to, carbohydrates having from 2 to 10 or more monosaccharides linked together (e.g., 2, 3, 4, 5, 6, 7, 8, 9, or 10 monosaccharides linked together). The constituent monosaccharide unit may be, for example, a pentose monosaccharide, a hexose monosaccharide, or a pseudosugar (including a pseudoamino sugar). Oligosaccharides do not include bicyclic groups that are formed by fusing a monosaccharide to a benzene ring, a cyclohexane ring, or a heterocyclic ring. Pseudosugars that may be used in the invention are members of the class of compounds wherein the ring oxygen atom of the cyclic monosaccharide is replaced by a methylene group. Pseudosugars are also known as “carba-sugars.”

The glycosyltransferases can catalyze addition of a monosaccharide to enasidenib, and the bond between the monosaccharide and enasidenib can be either an alpha or beta glycosidic bond. Disaccharides, trisaccharides, and oligosaccharides are formed by serial enzymatic additions of two or more monosaccharides to enasidenib. When more than one monosaccharide is added by serial enzymatic reactions, successive monosaccharides can be bonded to the preceding monosaccharide by either an alpha or beta glycosidic bond.

### Methods of Making Enasidenib Glycosides

Enasidenib glycosides can be made from enasidenib by an enzymatically catalyzed reaction. A reaction mixture is provided that includes enasidenib, a uridine diphosphate glycosyltransferase, and a uridine diphosphate-monosaccharide. After a period of time (e.g., from 1 to 72 hours), enasidenib is converted to a monosaccharide, disaccharide, trisaccharide, or oligosaccharide of enasidenib. The monosaccharide, disaccharide, trisaccharide, or oligosaccharide of enasidenib that is formed corresponds to the uridine diphosphate-monosaccharide that is included in the reaction mixture.

In some embodiments, the UGT enzyme and recombinant UGT-expressing cell lysate (e.g., yeast cell lysate) are placed in a reaction vessel. To form the lysate, UGT-expressing cells (e.g., UGT-expressing yeast cells) are lysed and the insoluble part is discarded by centrifugation so that the lysate is cell-free. In other embodiments, the cell-free lysate is not required. For example, in some embodiments, recombinant UGTs can be used. In other embodiments, purified UGTs can be used.

### Enasidenib Glycosides

Enasidenib glycosides are compounds represented by the following structural formula:

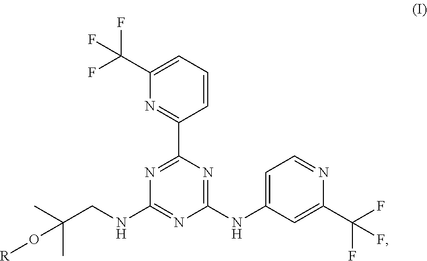

R is a monosaccharide, disaccharide, trisaccharide, or an oligosaccharide comprising 4 to 10 monosaccharides (e.g. 4, 5, 6, 7, 8, 9, or 10 monosaccharides). In some instances, the compound is a pharmaceutically acceptable salt of Compound (I).

In one embodiment, R is glucose, which can be D-glucose or L-glucose. D-glucose is represented by the following structural formula:

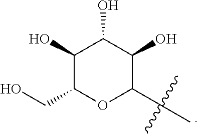 [SMILES: *C1O[C@H](CO)[C@@H](O)[C@H](O)[C@H]1O]

In one embodiment, R is galactose, which can be D-galactose or L-galactose. D-galactose is represented by the following structural formula:

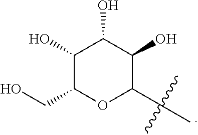 [SMILES: *C1O[C@H](CO)[C@H](O)[C@H](O)[C@H]1O]

In one embodiment, R is xylose, which can be D-xylose or L-xylose. Xylose can form six- and five-membered rings. A five-membered ring of D-xylose is represented by the following structural formula:

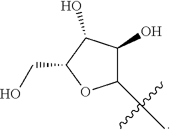 [SMILES: *C1O[C@H](CO)[C@H](O)[C@H]1O]

In one embodiment, R is N-acetylglucosamine, which can be D-N-acetylglucosamine or L-N-acetylglucosamine. D-N-acetylglucosamine is represented by the following structural formula:

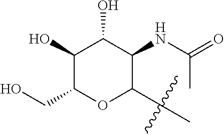 [SMILES: *C1O[C@H](CO)[C@@H](O)[C@H](O)[C@H]1NC(C)=O]

The bond between the monosaccharide (e.g., glucose) and enasidenib can be an alpha or beta glycosidic bond. The bond between monosaccharides of a disaccharide can be either an alpha or beta glycosidic bond. The bond between monosaccharides of a trisaccharide can be either an alpha or beta glycosidic bond. The bond between monosaccharides of an oligosaccharide can be either an alpha or beta glycosidic bond. The glycosidic bond between monosaccharides of a disaccharide or trisaccharide and between monosaccharides of an oligosaccharide can be formed between any of the hydroxyl groups from each monosaccharide. In other words, the bond between monosaccharides can be, e.g., 1→2, 1→3, 1→4, or 1→6.

In some embodiments, R is a disaccharide.

In one embodiment, R is a disaccharide consisting of two molecules of glucose, and the compound is enasidenib-di-O-D-glucoside. A disaccharide consisting of two monomers of glucose, where the two monomers are bonded by a 1→3 glycosidic bond, has the following structural formula:

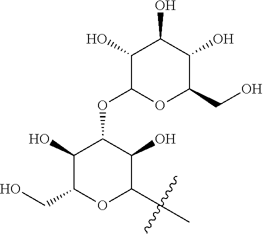 [SMILES: *C1O[C@H](CO)[C@@H](O)[C@H](OC2O[C@H](CO)[C@@H](O)[C@H](O)[C@H]2O)[C@H]1O]

In one embodiment, R is a disaccharide consisting of two molecules of galactose, and the compound is enasidenib-di-O-D-galactoside. A disaccharide consisting of two monomers of galactose, where the two monomers are bonded by a 1→3 glycosidic bond, has the following structural formula:

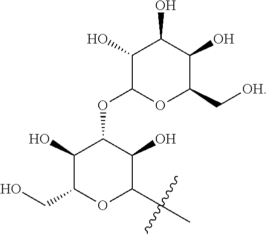 [SMILES: *C1O[C@H](CO)[C@@H](O)[C@H](OC2O[C@H](CO)[C@H](O)[C@H](O)[C@H]2O)[C@H]1O]

In one particular embodiment, R is a disaccharide consisting of two molecules of xylose, and the compound is enasidenib-di-O-D-xyloside. A disaccharide consisting of two monomers of xylose, where the two monomers are bonded by a 1→3 glycosidic bond, has the following structural formula:

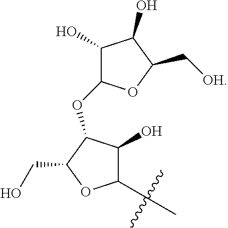 [SMILES: *C1O[C@H](CO)[C@H](OC2O[C@H](CO)[C@H](O)[C@H]2O)[C@H]1O]

In some embodiments, the disaccharide includes two different monosaccharides. In some embodiments, the trisaccharide or oligosaccharide includes two or more different monosaccharides. One example is enasidenib-O-xylose-glucoside.

In some embodiments, R is a trisaccharide.

In one embodiment, R is a trisaccharide consisting of three molecules of glucose, and the compound is enasidenib-tri-O-D-glucose. The three monomers are bonded by a 1→2, 1→3, or 1→4 glycosidic bond. A branched glucose trisaccharide with 1→3 and 1→2 bonds has the following structure:

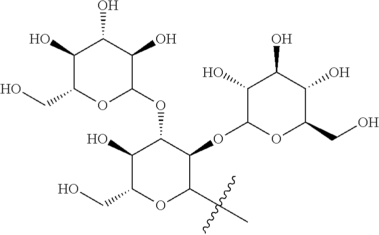 [SMILES: *C1O[C@H](CO)[C@@H](O)[C@H](OC2O[C@H](CO)[C@@H](O)[C@H](O)[C@H]2O)[C@H]1OC1O[C@H](CO)[C@@H](O)[C@H](O)[C@H]1O]

A branched glucose trisaccharide with 1→3 and 1→4 bonds has the following structure:

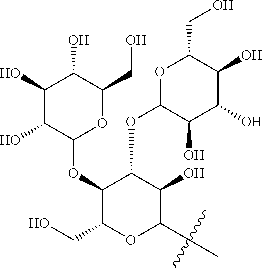 [SMILES: *C1O[C@H](CO)[C@@H](OC2O[C@H](CO)[C@@H](O)[C@H](O)[C@H]2O)[C@H](OC2O[C@H](CO)[C@@H](O)[C@H](O)[C@H]2O)[C@H]1O]

### Methods of Treating Diseases

The enasidenib glycosides described herein can be used in methods of treating diseases. The enasidenib glycoside is administered to a patient in need thereof.

Diseases that can be treated by administering the enasidenib glycosides disclosed herein include, but are not limited to, acute myeloid leukemia, glioma, chondrosarcoma, angioimmunoblastic T-cell lymphoma cancers, intrahepatic cholangiocarcinoma, cartilaginous tumors, precancerous diseases such as myelodysplastic syndrome, D-2-hydroxyglutaric aciduria, and Ollier disease, and Maffucci syndrome.

Typically, patients in need thereof have an isocitrate dehydrogenase-2 (IDH2) mutation, such as an R140 mutation (e.g., R140Q) and/or an R172 mutation (e.g., R172S, R172M, R172G, R172W, or R172K).

The enasidenib glycosides can be administered as part of a combination therapy.

One example of a combination therapy is administration of a hypomethylating agent, such as azacitidine and/or decitabine. Another example is co-administration of one or more standard cytotoxic therapeutic such as cytarabine, an anthracycline (e.g. daunorubicin or idarubicin), mitoxantrone, or etoposide. Other examples of a combination therapy include the co-administration of venetoclax, a B-cell lymphoma 2 (BCL-2) inhibitor; Telaglenastat (CB-839), a glutaminase inhibitor; FMS-like tyrosine kinase 3 (FLT-3) tyrosine kinase inhibitors such as midostaurin, lestaurtinib, and sunitinib; and/or all trans-retinoic acid, an inducer of differentiation.

The enasidenib glycosides described herein can be used in place of, or in addition to, enasidenib in those combination therapies.

### Pharmaceutical Compositions, Dosing, and Administration

Also provided herein is a pharmaceutical composition, comprising an enasidenib glycoside disclosed herein, or a pharmaceutically acceptable salt thereof, and optionally a pharmaceutically acceptable carrier. The compositions can be used in the methods described herein, e.g., to supply a compound described herein, or a pharmaceutically acceptable salt thereof.

“Pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some embodiments, an oral formulation is formulated for immediate release or sustained/delayed release.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the enasidenib glycosides of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants such as wetting agents, emulsifying and suspending agents, sweetening, flavoring, coloring, perfuming, and preservative agents.

Compositions suitable for buccal or sublingual administration include tablets, lozenges and pastilles, wherein the active ingredient is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

An enasidenib glycoside described herein can also be in micro-encapsulated form with one or more excipients, as noted above. In such solid dosage forms, the enasidenib glycoside can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the active ingredient against degradation as it passes through the alimentary tract, for example, by an outer coating of the formulation on a tablet or capsule.

In another embodiment, an enasidenib glycoside or pharmaceutically acceptable salt described herein can be provided in an extended (or “delayed” or “sustained”) release composition. This delayed-release composition includes the enasidenib glycoside or pharmaceutically acceptable salt in combination with a delayed-release component. Such a composition allows targeted release of a provided agent into the lower gastrointestinal tract, for example, into the small intestine, the large intestine, the colon and/or the rectum. In certain embodiments, a delayed-release composition further includes an enteric or pH-dependent coating, such as cellulose acetate phthalates and other phthalates (e.g., polyvinyl acetate phthalate, methacrylates (Eudragits)). Alternatively, the delayed-release composition provides controlled release to the small intestine and/or colon by the provision of pH sensitive methacrylate coatings, pH sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time-dependent hydrogel coatings and/or by acrylic acid linked to azoaromatic bonds coatings.

The amount of an enasidenib glycoside described herein, or a pharmaceutically acceptable salt thereof, that can be combined with the carrier materials to produce a composition in a single dosage form will vary depending upon the host treated, the particular mode of administration and the activity of the agent employed. Preferably, compositions should be formulated so that a dosage of from about 0.01 mg/kg to about 100 mg/kg body weight/day of the enasidenib glycoside, or pharmaceutically acceptable salt thereof, can be administered to a subject receiving the composition.

The desired dose may conveniently be administered in a single dose or as multiple doses administered at appropriate intervals such that, for example, the agent is administered 2, 3, 4, 5, 6 or more times per day. The daily dose can be divided, especially when relatively large amounts are administered, or as deemed appropriate, into several, for example 2, 3, 4, 5, 6 or more, administrations.

It should also be understood that a specific dosage and treatment regimen for any particular patient will depend upon a variety of factors, including the activity of the specific agent employed, the age, body weight, general health, sex, diet, time of administration, rate of excretion, drug combination, the judgment of the treating physician and the severity of the particular disease being treated. The amount of an enasidenib glycoside in the composition will also depend upon the particular enasidenib glycoside in the composition.

Other pharmaceutically acceptable carriers, adjuvants and vehicles that can be used in the compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be advantageously used to enhance delivery of agents described herein.

In some embodiments, compositions comprising an enasidenib glycoside described herein, or a pharmaceutically acceptable salt thereof, can also include one or more other therapeutic agents, e.g., in combination. When the compositions of this invention comprise a combination, the agents should be present at dosage levels of between about 1 to 100%, and more preferably between about 5% to about 95% of the dosage normally administered in a monotherapy regimen.

The compositions described herein can, for example, be administered by injection, intravenously, intraarterially, intraocularly, intravitreally, subdermally, orally, buccally, nasally, transmucosally, topically, in an ophthalmic preparation, or by inhalation, with a dosage ranging from about 0.5 mg/kg to about 100 mg/kg of body weight or, alternatively, in a dosage ranging from about 1 mg/dose to about 1000 mg/dose, every 4 to 120 hours, or according to the requirements of the particular drug. Typically, the compositions will be administered from about 1 to about 6 (e.g., 1, 2, 3, 4, 5 or 6) times per day or, alternatively, as an infusion (e.g., a continuous infusion). The amount of active ingredient that can be combined with a carrier material to produce a single dosage form will vary depending upon the host treated and the particular mode of administration. A typical preparation will contain from about 1% to about 95%, from about 2.5% to about 95% or from about 5% to about 95% of an enasidenib glycoside (w/w). Alternatively, a preparation can contain from about 20% to about 80% of an enasidenib glycoside (w/w).

Doses lower or higher than those recited above may be required. Specific dosage and treatment regimens for any particular patient will depend upon a variety of factors, including the activity of the specific agent employed, the age, body weight, general health status, sex, diet, time of administration, rate of excretion, drug combination, the severity and course of the disease, condition or symptoms, the patient's disposition to the disease, condition or symptoms, and the judgment of the treating physician.

“Treating,” as used herein, refers to taking steps to deliver a therapy to a subject, such as a mammal, in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“A therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause adverse side effects or produces minimal adverse side effects.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a human. “Subject” and “patient” are used interchangeably herein.

An enasidenib glycoside described herein, or a pharmaceutically acceptable salt thereof, can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the enasidenib glycoside and the particular disease to be treated. Administration can be local or systemic as indicated. The preferred mode of administration can vary depending on the particular enasidenib glycoside chosen.

Certain methods further specify a delivery route such as intravenous, intramuscular, subcutaneous, rectal, intranasal, pulmonary, or oral.

An enasidenib glycoside described herein, or a pharmaceutically acceptable salt thereof, can also be administered in combination with one or more other therapies (e.g., radiation therapy, a chemotherapy, such as a chemotherapeutic agent; an immunotherapy, such as an immunotherapeutic agent). When administered in a combination therapy, the enasidenib glycoside, or pharmaceutically acceptable salt thereof, can be administered before, after or concurrently with the other therapy (e.g., radiation therapy, an additional agent(s)). When co-administered simultaneously (e.g., concurrently), the enasidenib glycoside, or pharmaceutically acceptable salt thereof, and other therapy can be in separate formulations or the same formulation. Alternatively, the enasidenib glycoside, or pharmaceutically acceptable salt thereof, and other therapy can be administered sequentially, as separate compositions, within an appropriate time frame as determined by a skilled clinician (e.g., a time sufficient to allow an overlap of the pharmaceutical effects of the therapies).

In some embodiments, a method described herein further includes administering to the subject a therapeutically effective amount of an additional therapy (e.g., a hypomethylating agent, such as azacitidine and/or decitabine).

### SUMMARY

Enasidenib is a lipophilic, low solubility, high impact therapeutic that could benefit from modification by glycosylation.

There is a need for IDH inhibitors with improved aqueous solubility and with different PK/PD profiles to provide potential improvements in potency towards inhibiting the activity of the IDH protein and enhanced therapeutic effects on AML, and other diseases associated with IDH dysfunction.

### EXEMPLIFICATION

### Example 1: Establishment of a Glycosyltransferase (GT) Library and Cell Lysate-Based Assay to Identify Drug-Modifying Glycosyltransferases

Although GTs are one of the largest enzyme families in nature, the natural substrate(s) of the majority of GTs is unknown. Therefore, to identify GTs that can use a non-native substrate such as enasidenib is a nontrivial effort. A screening strategy was designed to address this need. The phylogenetic method was utilized to select a set of enzymes representing the structural and functional biodiversity of a desired functional GT class, uridine diphosphate (UDP) glycosyltransferases (UGTs), across different kingdoms and species. Based on the bioinformatics analysis, 328 UGTs were selected, including enzymes from different species of bacteria, fungus, plants, and human. To establish the GT library, the cDNA of the selected UGTs were produced by either nucleotide synthesis or by RT-PCR from the RNA of tissues expressing the UGTs. Each of the resulting UGT gene cDNA was cloned into the yeast TEF-promoter expression plasmid p426-TEF. The plasmids were individually transformed into wild-type yeast (Saccharomyces cerevisiae) strain BY4743. After auxotrophic selection, the yeast colonies expressing the recombinant UGT proteins were cultured, harvested, and lysed by CelLytic Y cell lysis reagent (Sigma-Aldrich). A cell-free cell lysate-based glycosylation assay utilizing the cell lysates was designed to screen for UGTs that are able to glycosylate the target substrate (see below for details). All UGTs were assayed in parallel on 96-well plates to allow for high throughput screening. The drug-modifying UGTs can be identified by the appearance of new peaks in HPLC analysis. The characteristics of the novel drug glycosides can be evaluated further by specialized assays.

### Example 2: Synthesis of enasidenib-O-D-glucoside and enasidenib-di-O-D-glucoside Using the Cell Lysate-Based Assay

A GT library made according to Example 1 was screened to identify enzymes able to catalyze regiospecific glycosylation of enasidenib when UDP-glucose was used as the sugar donor. Enasidenib (final concentration=50 μM) was added to each well of a 96-well microtiter plate containing a unique UGT enzyme in reaction mixture (50 mM Tris, pH 8.0, 10 mM UDP-glucose, and 20 μL recombinant UGT-expressing yeast cell lysate) and the reaction (total volume 100 μL) was allowed to proceed for 5 hours at 30° C., followed by termination of the modification reaction by quenching with 100 μL methanol. As a negative control, a reaction with the lysate of yeast harboring p426-TEF empty vector was carried out. The presence of the desired glycosylated product was determined by subjecting the contents of each well to HPLC analysis.

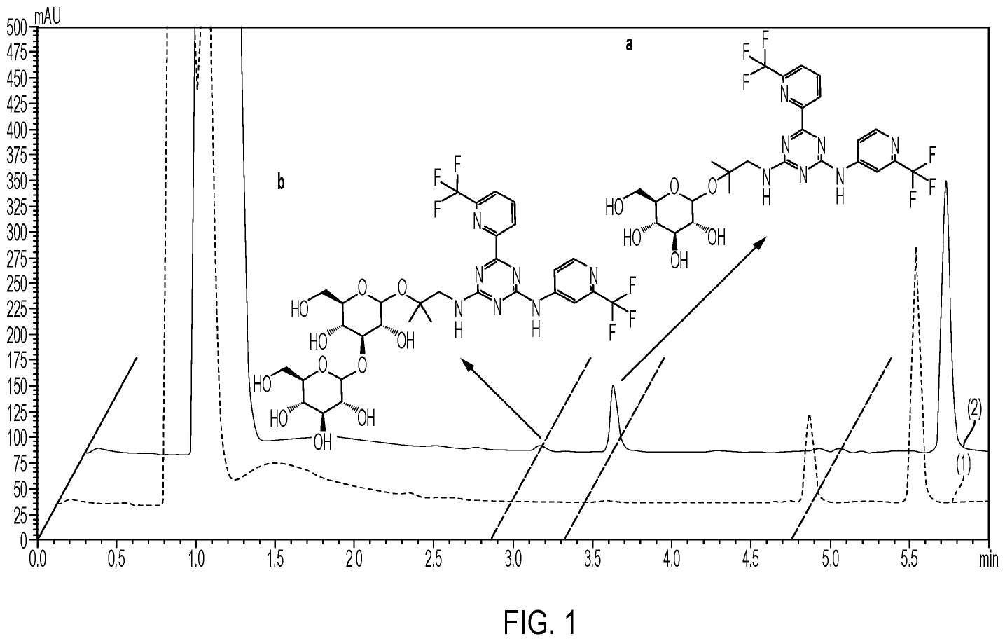

From the screen, three UGTs were able to modify enasidenib when UDP-glucose was used as the sugar donor. The overall conversion rates are: 96% for SEQ ID NO: 2, 34% for SEQ ID NO: 1, 9% for SEQ ID NO: 3. Among the three UGTs, SEQ ID NO: 1 and SEQ ID NO: 2 can produce both the monosaccharide enasidenib-O-D-glucoside (FIG. 1, chromatogram peak a) and the disaccharide enasidenib-di-O-D-glucoside (FIG. 1, chromatogram peak b). SEQ ID NO: 3 can produce enasidenib-O-D-glucoside only.

The chemical identity of the enasidenib glycosides was confirmed by LC-MS analysis: For a: m/z=636.25 [M+H]⁺; For b: m/z=798.19 [M+H]⁺.

The chemical identity of the enasidenib glycosides produced by SEQ ID NO: 1 was further confirmed by nuclear magnetic resonance (NMR) analyses: For a: ¹H NMR (DMSO-d₆, 400 MHz), δ 10.75 (1H, s), 8.70 (1H, m), 8.62 (1H, d, J=8.0 Hz), 8.55 (1H, m), 8.30 (1H, t, J=8.0 Hz), 8.10 (1H, d, J=7.6 Hz), 7.89 (1H, m), 4.40 (1H, d, J=7.6 Hz), 3.67 (1H, m), 3.58 (1H, d, J=6.0 Hz), 3.42 (1H, m), 3.18 (2H, m), 3.03 (3H, m), 1.23 (6H, s). For b: ¹H NMR (DMSO-d₆, 400 MHz), δ 10.69 (1H, s), 8.71 (1H, m), 8.61 (1H, d, J=8.0 Hz), 8.56 (1H, d, J=5.2 Hz), 8.28 (1H, m), 8.11 (1H, dd, J=7.6, 2.4 Hz), 7.82 (1H, m), 4.54 (1H, d, J=7.6 Hz), 4.45 (1H, d, J=7.6 Hz), 3.67 (2H, m), 3.53 (2H, m), 3.42 (2H, m), 3.22 (4H, m), 3.18 (2H, m), 3.12 (2H, m), 1.26 (6H, m).

The sequence of the enzymes identified as SEQ ID NOs.: 1-3 are disclosed herein in the sequences section.

### Example 3: Synthesis of enasidenib-O-D-glucoside, enasidenib-di-O-D-glucoside, and enasidenib-tri-O-D-glucoside Using Purified Recombinant Glycosyltransferases

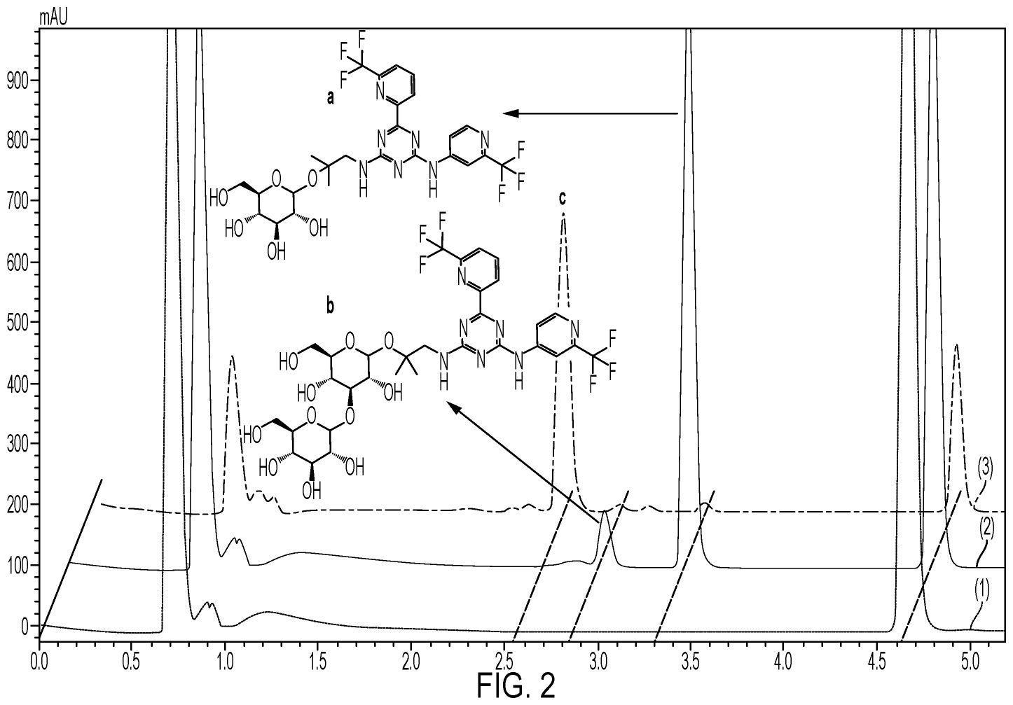

While a yeast cell lysate-based glycosylation assay is instrumental in initial screening efforts, one approach to producing larger amounts of ivacaftor glucosides is to use finely controlled enzyme concentrations during synthesis. To that end, two UGT genes identified in Example 2 (SEQ ID NO: 1 and 2) containing a metal-affinity purification tag at the C-terminus were transformed into BL21(DE3) Escherichia coli cells. Cells were grown at 37° C. until the cultures reached an optical density (OD600) of 0.5-0.8. Then, protein over-expression was induced with 1 mM isopropyl β-D-1-thiogalactopyranoside (IPTG) at 18° C. The culture was grown overnight for 16 hours and then harvested. Desired proteins were purified from the harvested cells using either free nickel-IDA resin or magnetic nickel-charged agarose beads. Ivacaftor glucoside synthesis using the purified recombinant enzymes was performed at volumes ranging from 10-75 mL. Enasidenib (final concentration 0.1 mg/ml) was added to the reaction mixture (final concentrations of 50 mM HEPES, 50 mM KCl, pH 7.5, 2 mM UDP-glucose, 1 uM UGT), and the reaction was allowed to proceed for 1-3 days at 37° C. The reaction was terminated by adding 1 reaction volume of ice-cold methanol. The reaction was then incubated at 90° C. to ensure that the enzyme was adequately denatured. The presence of the desired glycosylated product(s) was determined by HPLC analysis (FIG. 2).

The overall conversion rates are: 70% from SEQ ID NO: 1, and 97% from SEQ ID NO: 2. From these reactions, SEQ ID NO: 1 and 2 can produce the monosaccharide enasidenib-O-D-glucoside (FIG. 2 chromatogram peak a) and the disaccharide enasidenib-di-O-D-glucoside (FIG. 2. Chromatogram peak b). SEQ ID NO: 2 can also produce the trisaccharide enasidenib-tri-O-D-glucoside (FIG. 2 chromatogram peak c).

The chemical identity of the enasidenib glycosides was confirmed by LC-MS analysis: For a: m/z=636.09 [M+H]⁺; For b: m/z=798.11 [M+H]⁺; For c: 960.14 [M+H]⁺.

### Example 3: Synthesis of enasidenib-O-D-galactoside Using the Cell Lysate-Based Assay

A GT library made according to Example 1 was screened to identify enzymes able to catalyze regiospecific glycosylation of enasidenib when UDP-galactose was used as the sugar donor. Enasidenib (final concentration=50 μM) was added to each well of a 96-well microtiter plate containing a unique UGT enzyme in reaction mixture (50 mM Tris, pH 8.0, 2 mM UDP-galactose and 20 μL recombinant UGT-expressing yeast cell lysate) and the reaction (total volume 100 μL) was allowed to proceed for 5 hours at 30° C., followed by termination of the modification reaction by quenching with 100 μL methanol. As a negative control, a reaction with the lysate of yeast harboring p426-TEF empty vector was carried out. The presence of the desired glycosylated product was determined by subjecting the contents of each well to HPLC analysis.

From the screen, one UGT was able to modify enasidenib when UDP-galactose was used as the sugar donor. The overall conversion rates are: 8% for SEQ ID NO: 2. SEQ ID NO: 2 can produce monosaccharide enasidenib-O-D-galactoside.

The chemical identity of the enasidenib glycosides was confirmed by LC-MS analysis: m/z=636.22 [M+H]⁺.

### Example 4: Synthesis of enasidenib-O-D-xyloside Using Purified Recombinant Glycosyltransferases

The purified recombinant assay described in Example 3 was conducted with the following modification. UDP-xylose was used instead of UDP-glucose resulting in a final reaction mixture containing 50 mM HEPES, 50 mM KCl, pH 7.5, 2 mM UDP-xylose, 1 uM UGT, and 0.1 mg/ml enasidenib. The presence of the desired glycosylated product(s) was determined by HPLC analysis.

From this assay, SEQ ID NO: 1 and 2 were able to modify enasidenib when UDP-xylose was used as the sugar donor. The overall conversion rates are: 8% for SEQ ID NO: 1, and 31% for SEQ ID NO: 2. Both SEQ ID NO: 1 and 2 can produce monosaccharide enasidenib-O-D-xyloside.

The chemical identity of the etoposide glycoside was confirmed by LC-MS analysis: m/z=606.10 [M+H]⁺.

### Example 5: Comparison of the Water Solubility of enasidenib and enasidenib-O-D-glucoside

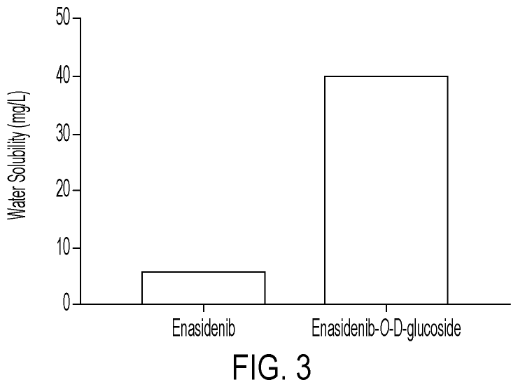

The water solubility of enasidenib and enasidenib-O-D-glucoside was investigated by suspending excess amounts of the two compounds in 200 μl of distilled water in a microcentrifuge tube at 25° C. for 12 h. Afterwards, each sample was centrifuged at 12,000×g for 20 min. The supernatant of each sample was then filtered through a 0.45-μm membrane filter and the concentration of the compound in the supernatant, which is defined as the water-soluble component, was measured by its absorbance at 254 nm using HPLC, and its absolute solubility was calculated in reference to the concentration-absorbance standard curve. As shown in FIG. 3, the water solubility of enasidenib was determined to be 6 mg/L, whereas that of enasidenib-O-D-glucoside was 40 mg/L, which is 7 times higher.

### REFERENCES

Abel, Mark, Roman Szweda, Daniel Trepanier, Randall W. Yatscoff, and Robert T. Foster. 2007. Rapamycin carbohydrate derivatives. USPTO U.S. Pat. No. 7,160,867 B2, filed May 13, 2004, and issued Jan. 9, 2007.

Alibhai, Shabbir M. H., Marc Leach, Mark D. Minden, and Joseph Brandwein. 2009. “Outcomes and Quality of Care in Acute Myeloid Leukemia over 40 Years.” Cancer 115 (13): 2903-11.

American Cancer Society. 2020. “Cancer Facts and FIGS. 2020.” Atlanta, Ga.: American Cancer Society.

Arts, Ilja C. W., Aloys L. A. Sesink, Maria Faassen-Peters, and Peter C. H. Hollman. 2004. “The Type of Sugar Moiety Is a Major Determinant of the Small Intestinal Uptake and Subsequent Biliary Excretion of Dietary Quercetin Glycosides.” The British Journal of Nutrition 91 (6): 841-47.

Berman, H. M. 2000. “The Protein Data Bank.” Nucleic Acids Research. https://doi.org/10.1093/nar/28.1.235.

Bonina, Francesco, Carmelo Puglia, Maria Grazia Rimoli, Daniela Melisi, Giampiero Boatto, Maria Nieddu, Antonio Calignano, Giovanna La Rana, and Paolo De Caprariis. 2003. “Glycosyl Derivatives of Dopamine and L-Dopa as Anti-Parkinson Prodrugs: Synthesis, Pharmacological Activity and in Vitro Stability Studies.” Journal of Drug Targeting 11 (1): 25-36.

Breton, Christelle, Sylvie Fournel-Gigleux, and Monica M. Palcic. 2012. “Recent Structures, Evolution and Mechanisms of Glycosyltransferases.” Current Opinion in Structural Biology 22 (5): 540-49.

Celgene Corp. 2017. “IDHIFA (enasidenib) [package Insert].” U.S. Food and Drug Administration. https://www.accessdata.fda.gov/drugsatfda_docs/label/2019/209606s002lbl.pdf.

De Bruyn, Frederik, Jo Maertens, Joeri Beauprez, Wim Soetaert, and Marjan De Mey. 2015. “Biotechnological Advances in UDP-Sugar Based Glycosylation of Small Molecules.” Biotechnology Advances 33 (2): 288-302.

Döhner, Hartmut, Anna Dolnik, Lin Tang, John F. Seymour, Mark D. Minden, Richard M. Stone, Teresa Bernal del Castillo, et al. 2018. “Cytogenetics and Gene Mutations Influence Survival in Older Patients with Acute Myeloid Leukemia Treated with Azacitidine or Conventional Care.” Leukemia 32 (12): 2546-57.

Döhner, Hartmut, Elihu H. Estey, Sergio Amadori, Frederick R. Appelbaum, Thomas Büchner, Alan K. Burnett, Hervé Dombret, et al. 2010. “Diagnosis and Management of Acute Myeloid Leukemia in Adults: Recommendations from an International Expert Panel, on Behalf of the European LeukemiaNet.” Blood 115 (3): 453-74.

Döhner, Hartmut, Daniel J. Weisdorf, and Clara D. Bloomfield. 2015. “Acute Myeloid Leukemia.” The New England Journal of Medicine 373 (12): 1136-52.

Fan, Bin, Yue Chen, Fang Wang, Katharine Yen, Luke Utley, Caroline Almon, Scott Biller, Sam Agresta, and Hua Yang. 2014. “Evaluation of Pharmacokinetic-Pharmacodynamic (PKPD) Relationship of an Oral, Selective, First-in-Class, Potent IDH2 Inhibitor, AG-221, from a Phase 1 Trial in Patients with Advanced IDH2 Mutant Positive Hematologic Malignancies.” Blood 124 (21): 3737-3737.

Fathi, Amir Tahmasb, Courtney Denton Dinardo, Irina Kline, Laurie Kenvin, Ira Gupta, Eyal C. Attar, Eytan M. Stein, Stéphane de Botton, and On Behalf of the AG-221-C-001 Study Investigators. 2017. “Differentiation Syndrome Associated with Enasidenib, a Selective Inhibitor of Mutant Isocitrate Dehydrogenase 2 (mIDH2).” Journal of Clinical Oncology 35 (15_suppl): 7015-7015.

Fernández, C., O. Nieto, E. Rivas, G. Montenegro, J. A. Fontenla, and A. Fernández-Mayoralas. 2000. “Synthesis and Biological Studies of Glycosyl Dopamine Derivatives as Potential Antiparkinsonian Agents.” Carbohydrate Research 327 (4): 353-65.

Galkin, Maria, and Brian A. Jonas. 2019. “Enasidenib in the Treatment of Relapsed/refractory Acute Myeloid Leukemia: An Evidence-Based Review of Its Place in Therapy.” Core Evidence 14 (April): 3-17.

Gantt, Richard W., Pauline Peltier-Pain, and Jon S. Thorson. 2011. “Enzymatic Methods for Glyco (diversification/randomization) of Drugs and Small Molecules.” Natural Product Reports 28 (11): 1811-53.

Gu, Xiangying, Lin Chen, Xin Wang, Xiao Liu, Qidong You, Wenwei Xi, Li Gao, et al. 2014. “Direct Glycosylation of Bioactive Small Molecules with Glycosyl Iodide and Strained Olefin as Acid Scavenger.” The Journal of Organic Chemistry 79 (3): 1100-1110.

Hardman, Janee' M., Robert T. Brooke, and Brandon J. Zipp. 2017. “Cannabinoid Glycosides: In Vitro Production of a New Class of Cannabinoids with Improved Physicochemical Properties.” bioRxiv. https://doi.org/10.1101/104349.

Huang, Chaoran, and Changfu Cheng. 2017. Deuterated compounds for treating hematologic malignancies, and compositions and methods thereof. U.S. Pat. No. 9,688,659 B2. Patent, filed Sep. 16, 2016, and issued Jun. 27, 2017.

Intlekofer, Andrew M., Alan H. Shih, Bo Wang, Abbas Nazir, Ariën S. Rustenburg, Steven K. Albanese, Minal Patel, et al. 2018. “Acquired Resistance to IDH Inhibition through Trans or Cis Dimer-Interface Mutations.” Nature 559 (7712): 125-29.

Krebber S., Claus, Christopher Davis, Stephen Delcardayre, Sergey A. Selifonov, and Russell Howard. 2001. Evolution and use of enzymes for combinatorial and medicinal chemistry. WIPO WO/2001/012817 A1. World Patent, filed Aug. 11,2000, and published Feb. 22, 2001.

Kr̆en, Vladimir. 2008. “Glycoside vs. Aglycon: The Role of Glycosidic Residue in Biological Activity.” Glycoscience, 2589-2644.

Kren, V., and L. Martínková. 2001. “Glycosides in Medicine: ‘The Role of Glycosidic Residue in Biological Activity.’” Current Medicinal Chemistry 8 (11): 1303-28.

Lairson, L. L., B. Henrissat, G. J. Davies, and S. G. Withers. 2008. “Glycosyltransferases: Structures, Functions, and Mechanisms.” Annual Review of Biochemistry 77 (1): 521-55.

Lin, Yih-Shyan, Rudeewan Tungpradit, Supachok Sinchaikul, Feng-Ming An, Der-Zen Liu, Suree Phutrakul, and Shui-Tein Chen. 2008. “Targeting the Delivery of Glycan-Based Paclitaxel Prodrugs to Cancer Cells via Glucose Transporters.” Journal of Medicinal Chemistry 51 (23): 7428-41.

Liu, Der-Zen, Supachok Sinchaikul, Peddiahgari Vasu Govardhana Reddy, Meng-Yang Chang, and Shui-Tein Chen. 2007. “Synthesis of 2′-Paclitaxel Methyl 2-Glucopyranosyl Succinate for Specific Targeted Delivery to Cancer Cells.” Bioorganic & Medicinal Chemistry Letters 17 (3): 617-20.

Lombard, Vincent, Hemalatha Golaconda Ramulu, Elodie Drula, Pedro M. Coutinho, and Bernard Henrissat. 2014. “The Carbohydrate-Active Enzymes Database (CAZy) in 2013.” Nucleic Acids Research 42 (D1): D490-95.

Medeiros, B. C., A. T. Fathi, C. D. DiNardo, D. A. Pollyea, S. M. Chan, and R. Swords. 2017. “Isocitrate Dehydrogenase Mutations in Myeloid Malignancies.” Leukemia 31 (2): 272-81.

Mellinghoff, Ingo, Marta Penas-Prado, Katherine Peters, Timothy Cloughesy, Howard Burris, Elizabeth Maher, Filip Janku, et al. 2018. “ACTR-31. PHASE 1 STUDY OF AG-881, AN INHIBITOR OF MUTANT IDH1 AND IDH2: RESULTS FROM THE RECURRENT/PROGRESSIVE GLIOMA POPULATION.” Neuro-Oncology 20 (suppl_6): vi18-vi18.

Mikuni, Katsuhiko, Katsuyoshi Nakanishi, Koji Hara, Kozo Hara, Wakao Iwatani, Tetsuya Amano, Kosho Nakamura, Yoshinori Tsuchiya, Hiroshi Okumoto, and Tadakatsu Mandai. 2008. “In Vivo Antitumor Activity of Novel Water-Soluble Taxoids.” Biological & Pharmaceutical Bulletin 31 (6): 1155-58.

NIH US National Library of Medicine. n.d. “ClinicalTrials.gov.” Accessed Jan. 27, 2020. http://clinicaltrials.gov.

Olthof, Margreet R., Peter C. H. Hollman, Tom B. Vree, and Martijn B. Katan. 2000. “Bioavailabilities of Quercetin-3-Glucoside and Quercetin-4′-Glucoside Do Not Differ in Humans.” The Journal of Nutrition 130 (5): 1200-1203.

Oran, B., and D. J. Weisdorf. 2012. “Survival for Older Patients with Acute Myeloid Leukemia: A Population-Based Study.” Haematologica 97 (12): 1916-24.

Peltier-Pain, Pauline, Shannon C. Timmons, Agnès Grandemange, Etienne Benoit, and Jon S. Thorson. 2011. “Warfarin Glycosylation Invokes a Switch from Anticoagulant to Anticancer Activity.” ChemMedChem 6 (8): 1347-50.

Polt, Robin, Muthu Dhanasekaran, and Charles M. Keyari. 2005. “Glycosylated Neuropeptides: A New Vista for Neuropsychopharmacology?” Medicinal Research Reviews 25 (5): 557-85.

Schmid, Jochen, Dominik Heider, Norma J. Wendel, Nadine Sperl, and Volker Sieber. 2016. “Bacterial Glycosyltransferases: Challenges and Opportunities of a Highly Diverse Enzyme Class Toward Tailoring Natural Products.” Frontiers in Microbiology 7 (February): 182.

Stein, Eytan M. 2016. “Molecular Pathways: IDH2 Mutations-Co-Opting Cellular Metabolism for Malignant Transformation.” Clinical Cancer Research: An Official Journal of the American Association for Cancer Research 22 (1): 16-19.

Stein, Eytan M., Courtney DiNardo, Jessica K. Altman, Robert Collins, Daniel J. DeAngelo, Hagop M. Kantarjian, Mikkael A. Sekeres, et al. 2015. “Safety and Efficacy of AG-221, a Potent Inhibitor of Mutant IDH2 That Promotes Differentiation of Myeloid Cells in Patients with Advanced Hematologic Malignancies: Results of a Phase 1/2 Trial.” Blood 126 (23): 323-323.

Stein, Eytan M., Courtney D. DiNardo, Daniel A. Pollyea, Amir T. Fathi, Gail J. Roboz, Jessica K. Altman, Richard M. Stone, et al. 2017. “Enasidenib in Mutant IDH2 Relapsed or Refractory Acute Myeloid Leukemia.” Blood 130 (6): 722-31.

Takechi, M., and Y. Tanaka. 1994. “Structure-Activity Relationships of Synthetic Digitoxigenyl Glycosides.” Phytochemistry 37 (5): 1421-23.

Terao, Junji, Sachiyo Yamaguchi, Mutsuko Shirai, Mariko Miyoshi, Jae-Hak Moon, Syunji Oshima, Takahiro Inakuma, Tojiro Tsushida, and Yoji Kato. 2001. “Protection by Quercetin and Quercetin 3-O-β-D-Glucuronide of Peroxynitrite-Induced Antioxidant Consumption in Human Plasma Low-Density Lipoprotein.” Free Radical Research 35 (6): 925-31.

Thol, Felicitas, Richard F. Schlenk, Michael Heuser, and Arnold Ganser. 2015. “How I Treat Refractory and Early Relapsed Acute Myeloid Leukemia.” Blood 126 (3): 319-27.

Torrens-Spence, Michael P., Tomáš Pluskal, Fu-Shuang Li, Valentina Carballo, and Jing-Ke Weng. 2018. “Complete Pathway Elucidation and Heterologous Reconstitution of Rhodiola Salidroside Biosynthesis.” Molecular Plant 11 (1): 205-17.

Waitkus, Matthew S., Bill H. Diplas, and Hai Yan. 2018. “Biological Role and Therapeutic Potential of IDH Mutations in Cancer.” Cancer Cell 34 (2): 186-95.

Walter, R. B., and E. H. Estey. 2015. “Management of Older or Unfit Patients with Acute Myeloid Leukemia.” Leukemia 29 (4): 770-75.

Xiao, Jianbo, Hui Cao, Yuanfeng Wang, Jinyao Zhao, and Xinlin Wei. 2009. “Glycosylation of Dietary Flavonoids Decreases the Affinities for Plasma Protein.” Journal of Agricultural and Food Chemistry 57 (15): 6642-48.

Xie, Kebo, Ridao Chen, Dawei Chen, Jianhua Li, Ruishan Wang, Lin Yang, and Jungui Dai. 2017. “EnzymaticN-Glycosylation of Diverse Arylamine Aglycones by a Promiscuous Glycosyltransferase from Carthamus tinctorius.” Advanced Synthesis & Catalysis 359 (4): 603-8.

Yen, Katharine, Jeremy Travins, Fang Wang, Muriel D. David, Erin Artin, Kimberly Straley, Anil Padyana, et al. 2017. “AG-221, a First-in-Class Therapy Targeting Acute Myeloid Leukemia Harboring Oncogenic IDH2 Mutations.” Cancer Discovery 7 (5): 478-93.

Yonekura-Sakakibara, Keiko, and Kousuke Hanada. 2011. “An Evolutionary View of Functional Diversity in Family 1 Glycosyltransferases.” The Plant Journal: For Cell and Molecular Biology 66 (1): 182-93.

Zhang, Yujiao, Kebo Xie, Aijing Liu, Ridao Chen, Dawei Chen, Lin Yang, and Jungui Dai. 2019. “Enzymatic Biosynthesis of Benzylisoquinoline Alkaloid Glycosides via Promiscuous Glycosyltransferases from Carthamus tinctorius.” Chinese Chemical Letters 30 (2): 443-46.

Zhu, Xiangming, and Richard R. Schmidt. 2009. “New Principles for Glycoside-Bond Formation.” Angewandte Chemie 48 (11): 1900-1934.

Zipp, Brandon Joel, Janee M. Hardman, and Robert T. Brooke. 2018. Cannabinoid glycoside prodrugs and methods of synthesis. USPTO US 2018/0264122 A1. U.S. Patent, published Sep. 20, 2018.

Altschul, S., Gish, W., Miller, W., Myers, E., and Lipman, D. (1990). Basic local alignment search tool. Journal of Molecular Biology. 215 (3): 403-4.

Henikoff, S. and J. G. Henikoff (1992). “Amino acid substitution matrices from protein blocks.” Proceedings of the National Academy of Sciences of the United States of America 89(22): 10915-10919.

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A compound represented by the following structural formula: 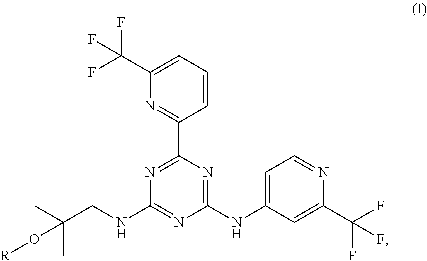
or a pharmaceutically acceptable salt thereof,
wherein R is a monosaccharide, a disaccharide, a trisaccharide, or an oligosaccharide comprising 4 to 10 monosaccharides.

2. The compound of claim 1, wherein R is a monosaccharide.

3. The compound of claim 2, wherein the monosaccharide is a pentose monosaccharide, hexose monosaccharide, or heptose monosaccharide.

4. The compound of claim 1, wherein R is allose, apiose, arabinose, fructose, fucitol, fucose, galactose, glucose, glucuronic acid, mannose, N-acetylglucosamine, N-acetylgalactosamine, rhamnose, or xylose.

5. The compound of claim 1, wherein R is glucosamine, galactosamine, mannosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, or D-glycero-D-altro-heptitol.

6. The compound of claim 1, wherein R is a disaccharide.

7. The compound of claim 6, wherein R is a disaccharide of two glucose molecules.

8. The compound of claim 6, wherein R is a disaccharide of two galactose molecules.

9. The compound of claim 6, wherein R is a disaccharide of two xylose molecules.

10. The compound of claim 6, wherein the disaccharide molecules are bonded by a 1→3 glycosidic bond.

11. The compound of claim 1, wherein R is a trisaccharide.

12. The compound of claim 11, wherein R is a trisaccharide of three glucose molecules.

13. The compound of claim 11, wherein R is a trisaccharide of three galactose molecules.

14. The compound of claim 11, wherein R is a trisaccharide of three xylose molecules.

15. The compound of claim 11, wherein the trisaccharide molecules are bonded by a 1→3 glycosidic bond and by a 1→2 glycosidic bond.

16. The compound of claim 11, wherein the trisaccharide has 1→3 and 1→4 glycosidic bonds.

17. (canceled)

18. A method of making an enasidenib glycoside, the method comprising:
a) providing a reaction mixture comprising:
i) a compound having the following structural formula: 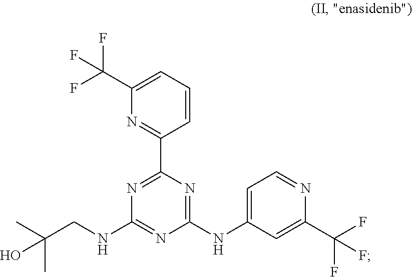 [SMILES: CC(C)(O)CNc1nc(Nc2ccnc(C(F)(F)F)c2)nc(-c2cccc(C(F)(F)F)n2)n1]
ii) a uridine diphosphate glycosyltransferase (UGT); and
iii) uridine diphosphate-monosaccharide;
b) allowing the reaction mixture to convert enasidenib to a monosaccharide, a disaccharide, a trisaccharide, or an oligosaccharide of enasidenib.

19. The method of claim 18, wherein the UGT comprises an amino acid sequence that is at least 95% similar to SEQ ID NO: 1.

20. The method of claim 18, wherein the UGT comprises an amino acid sequence that is at least 80% similar to a region from V278 to Q318 of SEQ ID NO: 1.

21. The method of claim 18, wherein the UGT comprises an amino acid sequence that is:
a) at least 90% similar to a region from I67 to D75 of SEQ ID NO: 1;
b) at least 90% similar to a region from D106 to L114 of SEQ ID NO: 1;
c) at least 90% similar to a region from C127 to S129 of SEQ ID NO: 1; and
d) at least 80% similar to a region from V278 to Q318 of SEQ ID NO: 1.

22. The method of claim 18, wherein the UGT comprises an amino acid sequence that is at least 95% similar to SEQ ID NO: 2.

23. The method of claim 18, wherein the UGT comprises an amino acid sequence that is at least 80% similar to a region from V291 to Q331 of SEQ ID NO: 2.

24. The method of claim 18, wherein the UGT comprises an amino acid sequence that is:
a) at least 90% similar to a region from W74 to V82 of SEQ ID NO: 2;
b) at least 90% similar to a region from D111 to V119 of SEQ ID NO: 2;
c) at least 90% similar to a region from F132 to N134 of SEQ ID NO: 2; and
d) at least 80% similar to a region from V291 to Q331 of SEQ ID NO: 2.

25. The method of claim 18, wherein the UGT comprises an amino acid sequence that is at least 95% similar to SEQ ID NO: 3.

26. The method of claim 18, wherein the UGT comprises an amino acid sequence that is at least 80% similar to a region from V283 to Q323 of SEQ ID NO: 3.

27. The method of claim 18, wherein the UGT comprises an amino acid sequence that is:
a) at least 90% similar to a region from I67 to Q79 of SEQ ID NO: 3;
b) at least 90% similar to a region from D110 to L118 of SEQ ID NO: 3;
c) at least 90% similar to a region from C131 to T133 of SEQ ID NO: 3; and
d) at least 80% similar to a region from V283 to Q323 of SEQ ID NO: 3.

28. The method of claim 18, wherein the uridine diphosphate-monosaccharide is uridine diphosphate-glucose (“UDP-glucose”).

29. The method of claim 18, wherein the uridine diphosphate-monosaccharide is uridine diphosphate-galactose (“UDP-galactose”).

30. The method of claim 18, wherein the uridine diphosphate-monosaccharide is uridine diphosphate-xylose (“UDP-xylose”).

31. The method of claim 18, wherein the uridine diphosphate-monosaccharide is uridine diphosphate-N-acetylglucosamine (“UDP-N-acetylglucosamine”).

32. A method of treating acute myeloid leukemia or an isocitrate dehydrogenase related disease, the method comprising administering to a patient in need thereof a therapeutically effective amount of a compound having the following structural formula: 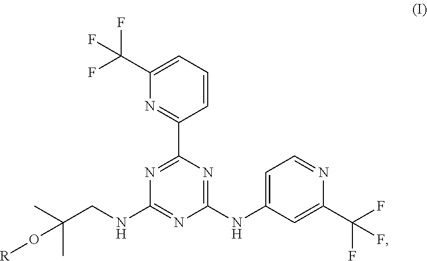
or a pharmaceutically acceptable salt thereof,
wherein R is a monosaccharide, a disaccharide, a trisaccharide, or an oligosaccharide comprising 4 to 10 monosaccharides.

33-35. (canceled)
